(19) United States
(12) Patent Application Publication
(10) Pub. No.: US 2023/0153874 A1
(43) Pub. Date: May 18, 2023

(54) BLOCKCHAIN BASED RADIOLOGY BILLING, RADIOLOGIST MANAGEMENT, AND RADIOLOGY DATA TRACKING SYSTEM

(71) Applicants: William Thoburn Randazzo, Augusta, GA (US); Eli Cornblath, Philadelphia, PA (US)

(72) Inventors: William Thoburn Randazzo, Augusta, GA (US); Eli Cornblath, Philadelphia, PA (US)

(21) Appl. No.: 17/987,544

(22) Filed: Nov. 15, 2022

Related U.S. Application Data
(60) Provisional application No. 63/279,848, filed on Nov. 16, 2021.

(51) Int. Cl. G06Q 30/04; G16H 30/20; G16H 50/70

(52) U.S. Cl. CPC G06Q 30/04; G16H 30/20; G16H 50/70

(57) ABSTRACT

A blockchain-based radiology billing, radiologist management, and radiology data tracking system that generates non-fungible tokens on the blockchain based on data retrieved from radiology workflows in the medical record for use in a marketplace. This invention may include a non-transitory computer-readable medium that records relevant data from the radiology information system, picture archiving and communication system, and electronic medical record, sorts and stores the data as metadata, transmits the sorted and stored metadata into a mint function on a smart contract on the blockchain, executes the creation of non-fungible tokens on the blockchain based on the metadata, imports the minted NFT into a marketplace, and allows a user to interface with the minted NFT on the marketplace. The marketplace enables collection of payment for radiology clinical services provided, payment for clinical radiology services provided, and tracking of clinical radiology work performed.

### CROSS-REFERENCE TO RELATED APPLICATION

This Patent Application claims priority to U.S. Provisional Patent Application No. 63/279,848, filed on Nov. 16, 2021, and entitled “Creation of non-fungible tokens on the blockchain using data from radiology information systems and picture archiving and communications systems for use in a marketplace.” The disclosure of the prior Application is considered part of and is incorporated by reference into this Patent Application.

### PRIOR ART

U.S. Pat. No. 11,443,838 B1, Cordonnier et al., September 2022

US 20220270146 A1, Diedrich et al., August 2022

U.S. Pat. No. 11,423,176 B2, Rosenberg et al., August 2022

US 20190180862 A1, Wisser et al., August 2022

US 20210342909 A1, Ketchel, May 2022

US 20220092566 A1, Austring and Hill, March 2022

US 20220093225 A1, Austring and Hill, March 2022

US 20210073877 A1, Mahadevan, March 2022

US 20220068502 A1, Yuz, March 2022

US 20220068464 A1, Yuz et al., March 2022

US 20220051276 A1, Zelocchi, February 2022

US 20210209688 A1, Krishnamurthy and Sharma, July 2021

U.S. Pat. No. 11,025,626 B1, Todd and O'Connell, June 2021

U.S. Pat. No. 11,017,123 B1, Salem, May 2021

US 20210098091 A1, Gomes et al., April 2021

US 20210057064 A1, Ballard et al., February 2021

US 20210012326 A1, Ashford and Zelocchi, January 2021

US 20210027885 A1, Averbach et al., January 2021

US 20200402629 A1, Austring et al., December 2020

US 20200388359 A1, Bhandari and Jain, December 2020

US 20200365243 A1, Swisher et al., November 2020

US 20200372983 A1, Zhang et al., November 2020

US 20220076794 A1, Averbach et al., October 2020

US 20200327616 A1, Burke et al., October 2020

US 20200327978 A1, Fower, October 2020

US 20200143084 A1, Rosenberg et al., October 2020

US 20200258605 A1, Blechman, August 2020

US 20190214116 A1, Eberting, July 2019

US 20190172572 A1, Piron et al., June 2019

US 20180350451 A1, Ohnemus et al., December 2018

US 20180294047 A1, Hosseini et al., October 2018

US 20180165588 A1, Saxena et al., June 2018

US 20180046766 A1, Deonarine et al., February 2018

US 20170329922 A1, Eberting et al., November 2017

### BACKGROUND

### Field of the Invention

This invention relates to a radiology billing and radiologist management system that utilizes information from the radiology workflow to generate billing and payment invoices as well as clinical-work-related data points on a distributed ledger (blockchain) as non-fungible tokens (NFTs) that may then be used to collect payment for clinical radiology services provided, pay for clinical radiology services provided, and track clinical radiology work performed. More particularly, this invention relates a non-transitory computer-readable medium that provides an interface for retrieving information from the medical record relevant to radiology billing and clinical radiology work performed, transmitting and storing the information retrieved from the medical record onto the blockchain as an NFT, and creating a marketplace for submitting and paying radiology invoices and tracking clinical radiology work performed.

### The Prior Art

Workflow in a radiology department and practice includes a radiology information system (RIS), picture archiving and communication system (PACS), and electronic medical record (EMR). The RIS and EMR contain information related to patient information, patient demographics, image type (i.e., imaging modality), body part, patient diagnosis/condition, and assignment of an image for interpretation by a radiologist, in addition to other details related to the imaging performed on a specific patient. The RIS is the graphical user interface (GUI) where radiologists interact with each patient file to open a radiology image and subsequently transmit an interpretation of the image (i.e., the radiology report) to the RIS, PACS, and EMR. PACS is the image viewing software that displays the patient's image and a limited amount of data tagging each image to a specific patient; the purpose of the PACS is to allow the radiologist to interact with and manipulate the image to render a clinical interpretation.

After completing the review of an imaging study, a billing workflow is initiated, which generally entails gathering patient information and information related to the type of imaging study performed.

Broadly, there are two types of billing workflows in radiology: preliminary/cash payment and final/insurance payment. In teleradiology, radiologists work remotely from the hospital or imaging center where images are acquired and are connected to the RIS, PACS, and EMR via the internet. In teleradiology, “preliminary” interpretations may be rendered by a radiologist, in which case billing is a peer-to-peer remittance that does not involve insurance vendors. However, in “preliminary” billing, a “final” interpretation is eventually provided by a different radiologist, usually working on site. When a “final” interpretation is provided as a stand-alone interpretation or following a “preliminary” interpretation, the billing pathway is generally directly through insurance companies without the intermediate step of remitting peer-to-peer payment. In some cases, payment for radiology services are performed as a peer-to-peer cash transaction for a “final” interpretation when agreed upon by both parties.

The preliminary billing workflow briefly entails creating a word document or spreadsheet annotating the radiology services provided and transmitting that document to the client via mail, facsimile, or electronic mail. The paying client reviews the submitted documentation and then remits payment to the radiology billing entity or radiology practice that provided the preliminary interpretation via check by mail or electronic funds transfer (EFT) via traditional banks. This process is labor intensive, prone to error, and has long turnaround times (up to 45 days or greater from when the bill is submitted to the payer).

The insurance-based workflow briefly entails collecting patient information, diagnosis, and imaging modality, which is then submitted to the insurer for eventual payment. Routinely, a third-party service is used for insurance related billing, which may be a dedicated department or separate company used by a hospital or radiology practice, which adds cost and labor to the billing process. The billing information is then supplied to another intermediary before the insurer, which depending upon the insurance company and other variables include entities such as an insurance clearing house, who are responsible for taking the submitted billing data (patient information, imaging modality, diagnosis, etc.) and compiling it into a format acceptable by the insurance company. This process also has a long turnaround time (weeks to months), is prone to error (both unintentional and intentional, i.e., insurance fraud), and requires many intermediate steps which add time, cost, and labor.

Related to billing payers for work performed by a radiologist, the radiologist is also paid via certain pathways, which rely directly upon receiving payment from the payers. Generally, once payment is collected via a preliminary/cash or final/insurance pathway, the radiologist is paid for the work performed. As above, the long turnaround time for collecting payment affects the time in which the radiologist is paid (i.e., there is a lag time in payment for the work performed). When paid, radiologists typically receive payment via EFT or check.

In the case of teleradiology, radiologists can be located worldwide and perform their clinical work (interpretation of radiology images) via a computer and an internet connection; specifically, they can receive radiology images and transmit their interpretation no matter where they are physically located in the world. Payment to the radiologist in these circumstances becomes more complex, particularly when the paying entity and radiologist are located in different countries (i.e., there is increased cost, time, and complexity with cross border fiat payments).

Lastly, radiologists interpret thousands to tens of thousands of separate imaging studies per year, which among other aspects, creates a large set of data useful for analyzing radiologist performance and productivity over time. Recording of this data is a labor-intensive process that requires unique and separate queries into the RIS, PACS, and EMR, depending on the data points that are sought.

In these respects, the proposed invention of a radiology billing and radiologist management system based on the creation of NFTs provides a single solution for retrieving information from the medical record, connecting payers and payees on a single marketplace, and for tracking radiologist performance.

### SUMMARY

This patent discloses and claims a novel, unobvious, and practical invention for radiology billing and radiologist management for collecting payment for clinical services provided, paying for clinical services provided, and tracking clinical radiology work performed.

To attain this, the present invention generally comprises a non-transitory computer-readable medium that may include one or more instructions that, when executed by one or more processors of a device, cause the device to: record data from the RIS, PACS, and/or EMR (referred to as “mined data”); sort and store the mined data in an array as metadata in a readable format for the blockchain; transmit the sorted and stored metadata into a mint function on a smart contract on the blockchain; execute the creation of a non-fungible token (NFT) on the blockchain based on the metadata; import the minted NFT into a marketplace; display the minted NFT along with the associated metadata on the marketplace for payers and payees to collect or remit payments and for clinical radiology data tracking. Other embodiments of this aspect include corresponding computer systems, apparatus, and computer programs recorded on one or more computer storage devices, each configured to perform the actions of the methods.

This invention may include a system of one or more computers configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. Implementations of the described techniques may include hardware, a method or process, or a computer tangible medium.

Implementations of this invention may include one or more of the following features. In one general aspect, an application programming interface (API) communicates with the RIS, PACS, and EMR for the purpose of recording data relevant to radiology billing and radiologist work performed. This data is typically contained in proprietary, firewall protected electronic health systems, which requires a purpose-built API to access the electronic health systems in a Health Insurance Portability and Accountability Act (HIPPA) compliant manner for securely interacting with protected patient information and for retrieving data relevant to the purpose of this invention, comprising radiology billing, radiology payment, and radiologist management, the specific data of which is disclosed in more detailed in the Detailed Description section and also referred to as “mined data” for descriptive purposes.

Once the mined data is identified and retrieved, an automated software algorithm sorts and stores the mined data based each imaging case, patient, and/or health care practitioner so it may be easily viewed, assessed, and tabulated by the user. The sorted and stored mined data is then uploaded into a mint function of a smart contract on the blockchain by the user, which allows for the creation of a non-fungible token (NFT).

Briefly, a smart contract is an electronic contract that is recorded on the blockchain and coded to perform specific functions (the smart contract functions relevant to this invention are described in the Detailed Description section). Briefly, an NFT is a token on the blockchain that is unique and may contain data (metadata) associated with the token. The metadata may be directly stored on the blockchain or on a centralized or decentralized database with the corresponding metadata labeled by the unique NFT number. Additionally, an NFT may be associated with a user uploaded image, which may be stored directly on the blockchain or on a centralized or decentralized database.

After the metadata is uploaded into the mint function, the user may then execute the creation of the NFT via a self-custody wallet associated with the blockchain, which requires the user to submit a “mint” transaction to the blockchain. Once the transaction is complete, the NFT is created and viewable on the blockchain as an immutable token. Additionally, the uploaded metadata is also viewable on the blockchain and associated with the minted NFT. If an image is also associated with the NFT, it may be viewable directly on the blockchain or viewed by accessing the image file location recorded in the metadata.

A separate API, denoted as the marketplace-API for descriptive purposes, queries the minted NFTs and associated metadata/image from the blockchain, and then displays the NFT and metadata/image on the marketplace graphical user interface (GUI). The NFTs for this invention may comprise several categories depending upon the uploaded metadata including but not limited to: invoices for radiology services provided, payment vouchers for radiology services performed, and data points for radiologist work performed. The invoices for radiology services provided may be paid by the clients by “buying” the NFT on the marketplace using cryptocurrency; the payment vouchers for radiology services performed may be “sold” for cryptocurrency on the marketplace to the payer; the data points for radiologist work performed may be exchanged for cryptocurrency as a “work related bonus” or stored in the user's wallet for data tracking.

A first advantage of this invention is the creation of an automated pathway to record information from the medical record that can be directly stored as an NFT on a distributed ledger (blockchain).

A second advantage of this invention is the transformation of medical information relevant to radiology billing, radiology payment, and radiologist work that is typically stored as a string of alphanumeric data and image files on proprietary, siloed system (as in a RIS, PACS, and EMR) into a new state as an NFT, which is auditable, immutable, and transferrable in a trustless, peer-to-peer manner on the blockchain.

A third advantage of this invention is a peer-to-peer invoicing and payment marketplace that allows for fast and direct invoicing and payments of radiology work performed without the long turnaround times of typical billing and payment pathways previously described.

A fourth advantage of this invention is a peer-to-peer invoicing and payment system that is trustless (i.e., the smart contract coded on the blockchain is viewable and auditable by anyone and determines the validity and terms of transaction, not a human) and independent of fiat-based payment pathways that are complex, costly, and time consuming when dealing with cross-border payments as may be the case in teleradiology.

A fifth advantage of this invention is an automated pathway to collect information from the medical record that relates to the work performed by a radiologist, which may then be used to track metrics such as reading rates and quality outcomes that can be tied into compensation and bonuses via the marketplace.

These and other features of the invention will be more readily understood upon consideration of the attached figures and of the following detailed description of those drawings and the presently-preferred and other embodiments of the invention. Additionally, it is to be understood that this invention is capable of other embodiments and that the terminology and phrases used are for the purpose of describing the invention but should not be regarded as limiting.

### BRIEF DESCRIPTION OF THE FIGURES

The previously described features of this invention as well as other features are described in the Detailed Description section and graphically illustrated in the following flowcharts:

FIG. 1 illustrating a flowchart of the overall architecture, function, and data flow in accordance with this invention;

FIG. 2 illustrating a flowchart of the recording of relevant data (“mined data”) via an API from the medical record in accordance with this invention;

FIG. 3 illustrating a flowchart of the sorting and storing of the mined data by an automated-software algorithm into an array as metadata in accordance with this invention;

FIG. 4 illustrating a flowchart of the transmitting of the sorted and stored metadata into a mint function of a smart contract in accordance with this invention;

FIG. 5 illustrating a flowchart of the creation of an NFT based on the metadata in accordance with this invention;

FIG. 6 illustrating a flowchart of the importing and displaying of the NFT and associated metadata/image onto a marketplace in accordance with this invention;

FIG. 7 illustrating a flowchart of the user interfacing with the NFT via the marketplace GUI in accordance with this invention.

### DETAILED DESCRIPTION

The following description of exemplary embodiments of this invention is not intended to limit in any manner the scope of the invention to these exemplary embodiments, but instead to guide any individual skilled in the art to make and be able to use this invention.

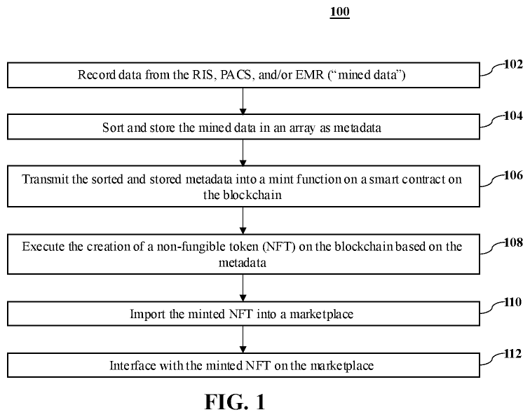

FIG. 1 is a flowchart of the overall architecture and process 100 of the invention, each process block of which is described in further detail in the additional figures. In some implementations, one or more process blocks of FIG. 1 may be performed by a non-transitory computer-readable medium.

As shown in FIG. 1, process 100 may include recording data from the RIS, PACS, and/or EMR (block 102), labeled as “mined data” for descriptive purposes. For example, a non-transitory computer-readable medium may record data from the RIS, PACS, and/or EMR, as described above. As also shown in FIG. 1, process 100 may include sorting and storing the mined data in an array as metadata (block 104). For example, a non-transitory computer-readable medium may sort and store the mined data in an array as metadata, as described above. As further shown in FIG. 1, process 100 may include transmitting the sorted and stored metadata into a mint function on a smart contract on the blockchain (block 106). For example, a non-transitory computer-readable medium may transmit the sorted and stored metadata into a mint function on a smart contract on the blockchain, as described above. As also shown in FIG. 1, process 100 may include executing the creation of a non-fungible token (NFT) on the blockchain based on the metadata (block 108). For example, a non-transitory computer-readable medium may execute the creation of an NFT on the blockchain based on the metadata, as described above. As further shown in FIG. 1, process 100 may include importing the minted NFT into a marketplace (block 110). For example, a non-transitory computer-readable medium may import the minted NFT into a marketplace, as described above. As also shown in FIG. 1, process 100 may include interfacing with the minted NFT on the marketplace (block 112). For example, a non-transitory computer-readable medium may interface with the minted NFT on the marketplace, as described above.

Although FIG. 1 shows example blocks of process 100, in some implementations, process 100 may include additional blocks, fewer blocks, different blocks, or differently arranged blocks than those depicted in FIG. 1. Additionally, or alternatively, two or more of the blocks of process 100 may be performed in parallel.

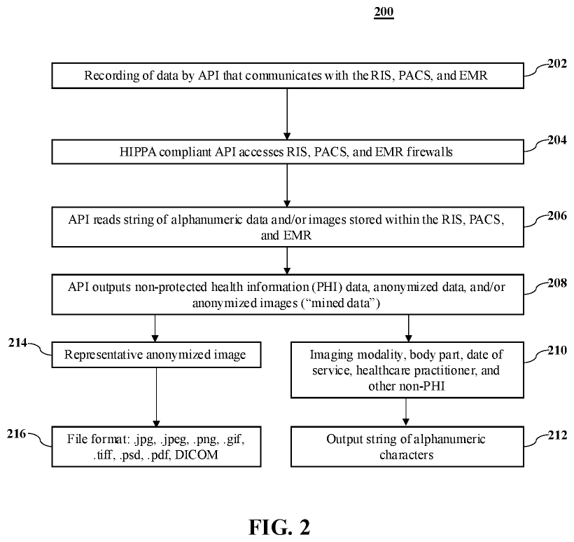

FIG. 2 is a flowchart in detail of the recording and retrieval process 200 of information from the medical record relevant to radiology billing, radiologist management, and clinical radiology data tracking. In some implementations, one or more process blocks of FIG. 2 may be performed by a non-transitory computer-readable medium.

Process 200 may include additional implementations, such as any single implementation or any combination of implementations described below and/or in connection with one or more other processes described elsewhere herein. In a first implementation, the recording of data is performed by an application programming interface (API) that communicates with the RIS, PACS, and EMR (block 202), which is purpose-built in order to meet the requirements of interfacing with medical records.

In a second implementation, alone or in combination with the first implementation, the API accesses the RIS, PACS, and EMR firewalls in a Health Insurance Portability and Accountability Act (HIPPA) compliant manner for securely interacting with protected patient information (block 204). This is an important aspect of the invention to ensure patient information and privacy is protected, and at minimum meets industry standards.

In a third implementation, alone or in combination with the first and second implementation, the API reads a string of alphanumeric data and/or images stored within the RIS, PACS, and EMR (block 206) relevant to radiology billing, radiology payment, and clinical radiology work performed.

In a fourth implementation, alone or in combination with one or more of the first through third implementations, the API outputs non-protected health information (PHI) data, anonymized data, and/or anonymized images (collectively referred to as “mined data” for descriptive purposes) (block 208) that is relevant to radiology billing, radiology payment, and clinical radiology work performed, described in further detail below.

In a fifth implementation, alone or in combination with one or more of the first through fourth implementations, the mined data may comprise imaging modality, body part, date of service, healthcare practitioner, and other non-PHI (block 210). Wherein the other non-PHI comprises age, gender, diagnosis, and medical condition/status.

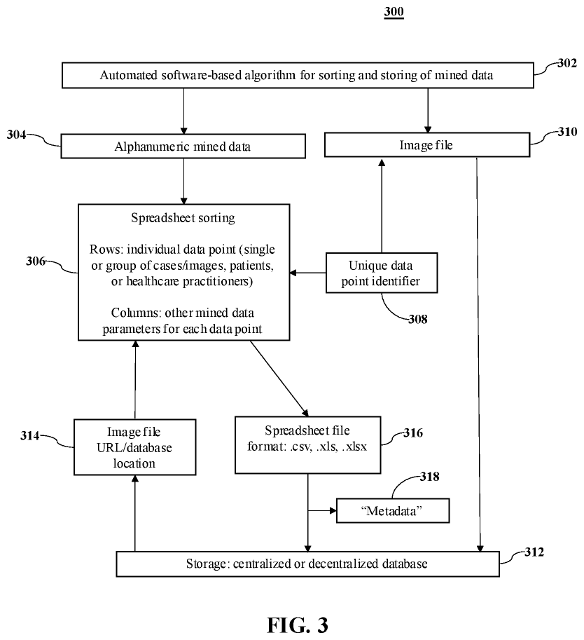

In a sixth implementation, alone or in combination with one or more of the first through fifth implementations, the output of the mined data from block 210 may comprise a string of alphanumeric characters (block 212) for further data processing described in FIG. 3 process 300 below.

In a seventh implementation, alone or in combination with one or more of the first through sixth implementations, the mined data may include a parallel pathway for recording and eventually storing a representative, anonymized image of the mined imaging modality (block 214).

In an eighth implementation, alone or in combination with one or more of the first through seventh implementations, the file format of the anonymized image from block 214 comprises .jpg, .jpeg, .png, .gif, .tiff, .psd, .pdf, and DICOM, among others (block 216)

Although FIG. 2 shows example blocks of process 200, in some implementations, process 200 may include additional blocks, fewer blocks, different blocks, or differently arranged blocks than those depicted in FIG. 2. Additionally, or alternatively, two or more of the blocks of process 200 may be performed in parallel.

FIG. 3 is a flowchart of the sorting and storing process 300 of the “mined data” (i.e., the API output described in FIG. 2 process 200), which is sorted into an array as metadata and stored onto centralized and/or decentralized databases. In some implementations, one or more process blocks of FIG. 3 may be performed by a non-transitory computer-readable medium. Process 300 may include additional implementations, such as any single implementation or any combination of implementations described below and/or in connection with one or more other processes described elsewhere herein. In a first implementation, the sorting and storing is performed by an automated software-based algorithm (block 302) designed to perform the actions outlined in blocks 304-316.

In a second implementation, alone or in combination with the first implementation, the automated software-based algorithm sorts the alphanumeric mined data (block 304: the API output of block 212 in FIG. 2), in a spreadsheet; the rows representing an individual data point (which may include a single or group of cases/images, patients, or healthcare practitioners); the columns representing the other mined data parameters for each data point retrieved from the RIS, PACS, and/or EMR (block 306).

In a third implementation, alone or in combination with the first and second implementation, a data point identifier is assigned to each row of the spreadsheet (in a designated column) as a unique number, not containing PHI or any association with identifying information in the medical record (block 308). Additionally, a unique data point identifier is assigned to the image file (block 310: the representative anonymized image obtained from the API output of block 214 FIG. 2). The unique data point identifier for each row of the spreadsheet matches the image file (block 310) data point identifier to associate the image file with the corresponding mined data obtained from the RIS, PACS, and/or EMR API output (block 210 FIG. 2).

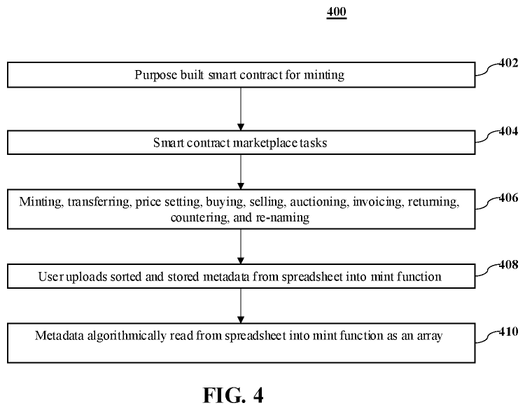

In a fourth implementation, alone or in combination with one or more of the first through third implementations, the image file (block 310) is stored on a centralized or decentralized database (block 312) for later use when uploading the data for NFT minting (described in FIG. 4 process 400).

In a fifth implementation, alone or in combination with one or more of the first through fourth implementations, the location of the image file, which may comprise a uniform resource locator (URL) when stored on a decentralized database or database file location when stored on a centralized database (block 314) is added to the spreadsheet file (block 306) in a designated column corresponding to the individual data point that the image file is associated with. This allows the user to know the location of the image file, once stored, and also allows the user to upload the correct image file linked to the corresponding metadata when uploading the data for NFT minting (described in FIG. 4 process 400).

In a sixth implementation, alone or in combination with one or more of the first through fifth implementations, the spreadsheet file format comprises .csv, .xls, and .xlsx, among others (block 316). The mined data, when sorted in the spreadsheet as described, will be termed “metadata” (block 318) for descriptive purposes of one embodiment of this invention; “metadata” is a term of art used for data associated with an NFT.

In a seventh implementation, alone or in combination with one or more of the first through eighth implementations, the spreadsheet is stored in a centralized or decentralized database (block 312) for later use when uploading the data for minting the NFT (described in FIG. 4 process 400).

Although FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel.

FIG. 4 is a flowchart of the uploading process 400 of the sorted and stored metadata (i.e., the mined data in the spreadsheet) into a mint function of a smart contract on the blockchain. In some implementations, one or more process blocks of FIG. 4 may be performed by a non-transitory computer-readable medium.

Process 400 may include additional implementations, such as any single implementation or any combination of implementations described below and/or in connection with one or more other processes described elsewhere herein. In a first implementation, a smart contract is written in the standard language of the blockchain utilized for minting (block 402), specifically coded and built for the purposes of this invention. Many different blockchains may be used to mint NFTs and the specific blockchain used to mint can vary and does not ultimately affect the functionality of this invention.

In a second implementation, alone or in combination with the first implementation, the smart contract is coded at minimum to perform tasks required in a marketplace (block 404). The marketplace tasks comprise minting, transferring, price setting, buying, selling, auctioning, invoicing, returning, countering, and re-naming (block 406).

In a third implementation, alone or in combination with the first and second implementation, the user uploads the sorted and stored metadata from the spreadsheet into a mint function (block 408). The image file URL/database location (FIG. 3, block 314) is used to represent the image in the mint function metadata, although in some embodiments of this invention, the image file may be uploaded directly into the mint function to be stored directly on the blockchain.

In a fourth implementation, alone or in combination with one or more of the first through third implementations, the alphanumeric metadata is algorithmically read from the spreadsheet into the mint function as an array formatted in a standard readable format for the blockchain (block 410). The mint function typically accepts alphanumeric data as an array, the desired order of which is coded into the smart contract (block 402); the order of the array may vary, ultimately not affecting the functionality of this invention.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

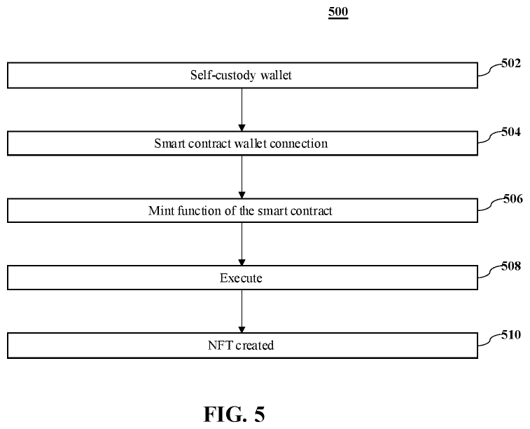

FIG. 5 is a flowchart of the NFT creation process 500 using the mint function of the smart contract (FIG. 4 block 402) described for this invention. In some implementations, one or more process blocks of FIG. 5 may be performed by a non-transitory computer-readable medium.

Process 500 may include additional implementations, such as any single implementation or any combination of implementations described below and/or in connection with one or more other processes described elsewhere herein. In a first implementation, the creation of the NFT is performed by the user's self-custody wallet (block 502) connecting to the smart contract built for this invention (FIG. 4 block 402) via the smart contract address. The smart contract address (a unique, automatically generated address assigned during the creation of the smart contract on the blockchain) is programmed into the wallet connection process (block 504) to ensure the user connects their self-custody wallet to the appropriate smart contract for minting related to this invention.

In a second implementation, alone or in combination with the first implementation, the self-custody wallet may be proprietary or based on existing software (block 502).

In a third implementation, alone or in combination with the first and second implementation, the self-custody wallet is used to interface with the mint function (block 506) and execute a mint transaction (block 508) to the blockchain via the specific smart contract built for this invention. Upon processing the mint transaction by the blockchain (which may be performed by proof of work, proof of stake, or other consensus mechanism as determined by the blockchain used for minting), the NFT is created (block 510) and viewable on the blockchain as an immutable, auditable token containing the metadata uploaded in process 400.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

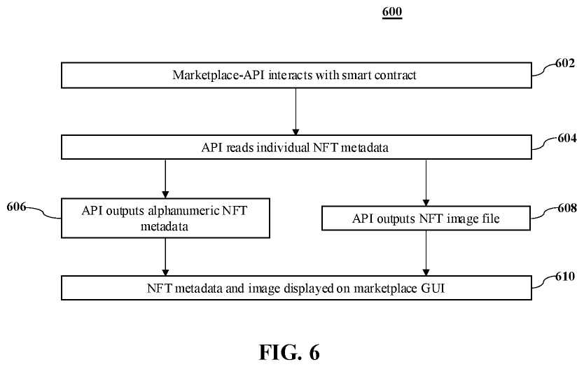

FIG. 6 is a flowchart of the marketplace-API process 600 that reads the NFT and associated metadata minted on the blockchain as described in process 500 in order to display the NFT and associated metadata on the marketplace GUI. In some implementations, one or more process blocks of FIG. 6 may be performed by a non-transitory computer-readable medium.

Process 600 may include additional implementations, such as any single implementation or any combination of implementations described below and/or in connection with one or more other processes described elsewhere herein. In a first implementation, a marketplace-API interacts with the specific smart contract (block 602) used to mint the NFTs on the blockchain in process 500, which may comprise a read-only function to identify the correct smart contract by the specific contract address, as previously described in process 500.

In a second implementation, alone or in combination with the first implementation, the marketplace-API reads the individual NFT metadata (block 604), which includes the alphanumeric mined data and image file URL and/or database file location described in process 300.

In a third implementation, alone or in combination with the first and second implementation, the marketplace-API outputs the NFT metadata read from the blockchain of the smart contract onto the marketplace for viewing by the user (block 606), which comprises the alphanumeric mined data read into the mint function from the spreadsheet described in process 400.

In a fourth implementation, alone or in combination with one or more of the first through third implementations, the marketplace-API also retrieves and outputs the image file (block 608) obtained from the URL or database location information stored as metadata associated with each NFT (as described in process 300). This allows the user to visualize an image for each NFT. Although, in some embodiments of this invention, the image file may be read directly from the blockchain, if uploaded directly into the mint function as described in process 400.

In a fifth implementation, alone or in combination with one or more of the first through fourth implementations, the alphanumeric metadata and image file are displayed on the marketplace graphical user interface (GUI) (block 610) for the user to interact with as described below in process 700.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

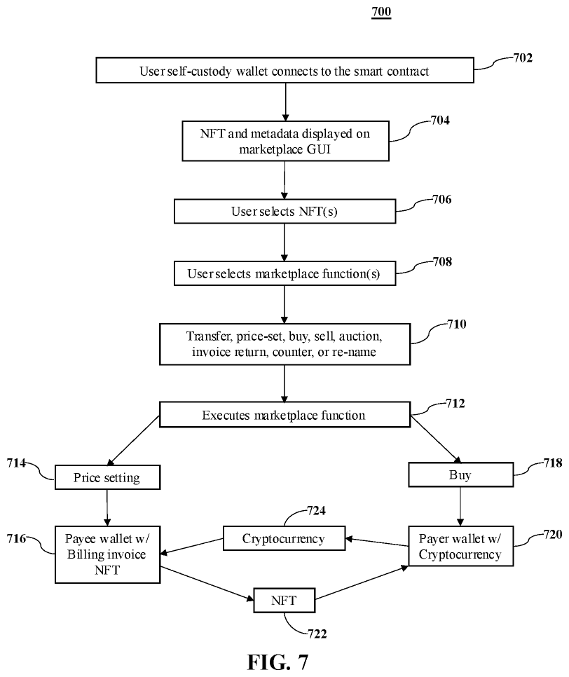

FIG. 7 is a flowchart of the marketplace GUI process 700 that enables the user to interface and interact with the minted NFT and associated metadata in order to perform functions relevant to this invention. In some implementations, one or more process blocks of FIG. 7 may be performed by a non-transitory computer-readable medium.

Process 700 may include additional implementations, such as any single implementation or any combination of implementations described below and/or in connection with one or more other processes described elsewhere herein. In a first implementation, the user's self-custody wallet is used to connect to the marketplace GUI via the specific smart contract address (block 702, also described in process 500), which may then initiate the individual NFTs to display as discrete items with the associated metadata and image file (block 704).

In a second implementation, alone or in combination with the first implementation, the self-custody wallet may be proprietary or based on existing software.

In a third implementation, alone or in combination with the first and second implementation, the user may select an individual NFT or multiple NFTs (block 706) as well as a marketplace function or functions (block 708), which are coded into the smart contract used for this invention (FIG. 4 block 402). Marketplace functions comprise transferring, price-setting, buying, selling, auctioning, invoicing, returning, countering, and re-naming (block 710). These marketplace functions allow the users to determine the pathway for the created NFT(s), which comprise the core functionality of this invention: collecting payment for clinical radiology services provided, paying for clinical radiology services provided, and track clinical radiology work performed.

In a fourth implementation, alone or in combination with one or more of the first through third implementations, the user executes the selected marketplace function by submitting a transaction to the blockchain via a self-custody wallet connected to the marketplace (block 712). In regards to payment related marketplace functions comprising price-setting, buying, and selling, cryptocurrency of the native blockchain used for minting, or other cryptocurrencies stored in the user's self-custody wallet, may be used for these payment related functions and monetary transfer. In the example of invoicing a radiology bill, the price-setting marketplace function (block 714) is used to set a price of the NFT by the entity billing for radiology services provided. When price-setting is selected (block 710) the GUI of the marketplace enables the user to input a numeric value of no less than 0 (for the numeric price) and a type of cryptocurrency (for the invoice to be paid in) as configured on the marketplace GUI. Upon executing the price-setting marketplace function (blocks 712 and 714) by the payee who owns the billing invoice NFT in their wallet (block 716), a transaction is submitted to the blockchain and once confirmed, the marketplace GUI will then update the displayed price of the NFT(s) as determined by the user setting the price. Furthermore, in the example of a radiology billing invoice, the payer (block 720, typically a separate wallet containing cryptocurrency to pay for the NFT invoice) may execute the “buy” marketplace function (block 718), which enables an exchange of the NFT (block 722) for the corresponding value of cryptocurrency (block 724) the NFT price was set for, regulated by the smart contract. The transferred NFT (block 722) becomes a “receipt” for the payer stored in their wallet (block 720) and the payee (block 716) receives the correct value of cryptocurrency as payment. Other marketplace functions may execute via similar pathways and accommodate a plurality of user wallets performing multiple marketplace tasks in sequence or in parallel.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A non-transitory computer-readable medium storing a set of instructions for creating non-fungible tokens (NFTs) on a distributed ledger (blockchain) using data from radiology information systems (RIS), picture archiving and communication systems (PACS), and electronic medical records (EMR) for radiology billing, radiologist management, and radiology data tracking, the set of instructions comprising:
one or more instructions that, when executed by one or more processors of a device, cause the device to:
record data from the RIS, PACS, and/or EMR (mined data);
sort and store the mined data in an array as metadata
transmit the sorted and stored metadata into a mint function on a smart contract on the blockchain
execute the creation of a non-fungible token (NFT) on the blockchain based on the metadata
import the minted NFT into a marketplace
interface with the minted NFT on the marketplace.

2. The non-transitory computer-readable medium of claim 1, wherein the recording of data is performed by an application programming interface (API) that communicates with the RIS, PACS, and EMR.

3. The non-transitory computer-readable medium of claim 2, wherein the API accesses the RIS, PACS, and EMR firewalls in a Health Insurance Portability and Accountability Act (HIPPA) compliant manner for securely interacting with protected patient information.

4. The non-transitory computer-readable medium of claim 2, wherein the API reads an alphanumeric string of data and/or images stored within the RIS, PACS, and EMR.

5. The non-transitory computer-readable medium of claim 2, wherein the API outputs non-protected health information (PHI) data, anonymized data, and/or anonymized images (collectively referred to as mined data).

6. The non-transitory computer-readable medium of claim 5, wherein the mined data comprises imaging modality.

7. The non-transitory computer-readable medium of claim 6, wherein imaging modalities comprise: X-ray (XR), ultrasound (US), computed tomography (CT), magnetic resonance imaging (MRI), nuclear medicine imaging (NM), mammography, and cardiac imaging.

8. The non-transitory computer-readable medium of claim 5, wherein the mined data comprises an anonymized image of the mined imaging modality.

9. The non-transitory computer-readable medium of claim 8, wherein the file format of the image comprises .jpg, .jpeg, .png, .gif, .tiff, .psd, .pdf, and DICOM, among others.

10. The non-transitory computer-readable medium of claim 5, wherein the mined data comprises body part.

11. The non-transitory computer-readable medium of claim 5, wherein the mined data comprises date of service.

12. The non-transitory computer-readable medium of claim 5, wherein the mined data comprises health care practitioner.

13. The non-transitory computer-readable medium of claim 5, wherein the mined data comprises other non-PHI.

14. The non-transitory computer-readable medium of claim 13, wherein the other non-PHI comprises: age, gender, diagnosis, and medical condition/status.

15. The non-transitory computer-readable medium of claim 1, wherein the sorting and storing is performed by an automated software-based algorithm.

16. The non-transitory computer-readable medium of claim 15, wherein the automated software-based algorithm organizes alphanumeric mined data in a spreadsheet; the rows representing an individual data point (which comprises a single or group of cases/images, patients, or healthcare practitioners); the columns representing the other mined data parameters for each data point retrieved from the RIS, PACS, and/or EMR.

17. The non-transitory computer-readable medium of claim 16, wherein the spreadsheet file format comprises .csv, .xls, and .xlsx, among others.

18. The non-transitory computer-readable medium of claim 16, wherein the spreadsheet and image from the mined data may be stored on a centralized and/or decentralized database with each individual data point and corresponding image labeled with a data point identifier.

19. The non-transitory computer-readable medium of claim 18, wherein a data point identifier is a unique, assigned number, not containing PHI.

20. The non-transitory computer-readable medium of claim 1, wherein the user uploads the sorted and stored metadata from the spreadsheet and image file into a mint function.

21. The non-transitory computer-readable medium of claim 20, wherein the metadata is algorithmically read from the spreadsheet into the mint function as an array formatted in a standard readable format for the blockchain.

22. The non-transitory computer-readable medium of claim 1, wherein the smart contract is written in the standard language of the blockchain utilized for minting.

23. The non-transitory computer-readable medium of claim 1, wherein the smart contract is coded at minimum to perform tasks required in a marketplace.

24. The non-transitory computer-readable medium of claim 23, wherein the marketplace tasks comprise minting, transferring, price setting, buying, selling, auctioning, invoicing, returning, countering, and re-naming.

25. The non-transitory computer-readable medium of claim 1, wherein the creation of the NFT is performed by executing the mint function in the smart contract.

26. The non-transitory computer-readable medium of claim 1, wherein the user interfaces with the blockchain via a self-custody wallet in order to execute the mint function in the smart contract.

27. The non-transitory computer-readable medium of claim 26, wherein the self-custody wallet may be proprietary or based on existing software.

28. The non-transitory computer-readable medium of claim 1, wherein a marketplace-API reads and displays the metadata and image previously minted on the blockchain.

29. The non-transitory computer-readable medium of claim 28, wherein the marketplace-API interacts with the specific smart contract on the blockchain where the NFTs were minted and reads the individual NFT metadata and image file (mined data).

30. The non-transitory computer-readable medium of claim 28, wherein the marketplace-API outputs the NFT metadata and image file (mined data) read from the smart contract onto the marketplace for viewing by the user.

31. The non-transitory computer-readable medium of claim 1, wherein the graphical user interface (GUI) of the marketplace displays individual NFTs as discrete items and displays the associated metadata and image file (mined data).

32. The non-transitory computer-readable medium of claim 1, wherein the user functions of the marketplace GUI are coded in the smart contract and comprise transferring, price-setting, buying, selling, auctioning, invoicing, returning, countering, and re-naming.

33. The non-transitory computer-readable medium of claim 32, wherein the user interfaces with the blockchain via a self-custody wallet in order to execute the marketplace functions in the smart contract.

34. The non-transitory computer-readable medium of claim 33, wherein the self-custody wallet may be proprietary or based on existing software.
